(19) United States
(12) Patent Application Publication
(10) Pub. No.: US 2002/0182257 A1
(43) Pub. Date: Dec. 5, 2002

(54) Multi-photon imaging and quantum lithography

(75) Inventors: Yanhua Shih, Baltimore, MD (US); Milena D'Angelo, Barry (IT); Maria Chekhova, Moscow (RU)

Correspondence Address: SUGHRUE MION, PLLC, 2100 Pennsylvania Avenue, NW, Washington, DC, 20037-3213, US

(73) Assignee: UNIVERSITY OF MARYLAND

(21) Appl. No.: 10/146,813

(22) Filed: May 17, 2002

Related U.S. Application Data
(60) Provisional application No. 60/292,265, filed on May 18, 2001.

(51) Int. Cl. A61K 9/14

(52) U.S. Cl. 424/489

(57) ABSTRACT

A microscopic image product comprising a light source that produces light that is made of entangled photons. Light made of entangled photons is used in lithography and other applications producing improved resolution for microscopic images.

### RELATED APPLICATIONS

This Application claims priority from co-pending U.S. Provisional Application Serial No. 60/292,265 filed May 18, 2001, which is incorporated in its entirety by reference.

### STATEMENT OF FEDERALLY SPONSORED RESEARCH

This invention was made with government support under grant no. N00014-91-J-1430 awarded by the Office of Naval Research. The government has certain rights in this invention.

### FIELD

This disclosure teaches techniques related to quantum entangled multi-photon states and their use in lithography and other applications. Specifically, systems and methods for performing lithography using quantum entangled light sources are disclosed. It should be noted that the techniques are applicable to any field where a conventionally classical light had been used for producing a microscopic image. The disclosed technique produces improved resolution.

### BACKGROUND

1. References

The following papers provide useful background information, for which they are incorporated herein by reference in their entirety, and are selectively referred to in the remainder of this disclosure by their accompanying reference codes in square brackets (i.e., <3>for the paper by M. O. Scully.):

<1>A. Einstein, B. Podolsky, and N. Rosen, Phys. Rev. 47, 777 (1935).

<2>A. N. Boto et al., Phys. Rev. Lett. 85, 2733 (2000).

<3>M. 0. Scully, in Proceedings of the Conference on Effects of Atomic Coherence and Interference in Quantum Optics, Crested Butte, Colorado, 1993 (IOP, Bristol, 1994); see also U. Rathe and M. O. Scully, Lett. Math. Phys. 34, 297 (1995).

<4>Y.-H. Kim and Y. H. Shih, Found. Phys. 29, 1849 (1999).

<5>See classical optics textbooks, for example, E. Hecht, Optics (Addison-Wesley, Reading, Mass., 1989), 2nd ed.

<6>T.B. Pittman et al., Phys. Rev. A 52, R3429 (1995); D. V. Strekalov et al., Phys. Rev. Lett. 74, 3600 (1995).

<7>D. N. Klyshko, Photons and Nonlinear Optics (Gordon and Breach Science, New York, 1988).

<8>A. Yariv, Quantum Electronics (John Wiley and Sons, New York, 1989).

<9>A.V. Burlakov, M. V. Chekhova, D. N. Klyshko, S. P. Kulik, A. N. Penin, Y. H. Shih, and D. V. Strekalov, Phys. Rev. A 56, 3214 (1997).

<10>T. E. Keller, M. H. Rubin, Y. H. Shih, and L. A. Wu, Phys. Rev. A 57, 2076 (1998).

<11>C. Fischer, Scanning Probe Microscopy, edited by R. Wiesendanger (Springer, New York, 1998), and references cited therein.

### INTRODUCTION

One of the principles of geometrical optics is that “light propagates in a straight line.” If this were always true, one could obtain the image of a physical object, for example, a physical slit, with an unlimited small size by applying a perfect lens system. However, light is also a wave. The minimum size of the image that can be created is determined by the wave property of light, namely, diffraction. The physics of diffraction is described herein.

According to the Huygens-Fresnel principle, each point on the primary wave front serves as the source of spherical secondary amplitudes (wavelets). These secondary amplitudes advance with the same speed and frequency as those of the primary wave. The wavelets, with different phases, from a physical slit will meet at any point in space. The superposition of the wavelets will determine the size of the image. The intensity distribution of light can be calculated by considering an integral of the wavelets coming from the physical object.

Consider a classical one-dimensional optical diffraction by a single slit. A well-collimated laser beam passes through the slit and then the intensity distribution of the beam is analyzed in the Fourier transform plane (or in the far-field zone). This distribution, which is the diffraction pattern of a single slit, is well known as:

sinc² (β), where

sinc (β)=sin(β)/β,

the parameter β=(πa/λ)θ, a is the width of the slit, and θ is the scattering angle <5>.

When β reaches π, the superposition of the wavelets results in a minimum intensity. The sinc²(β) pattern determines the minimum width one can obtain. Usually, this minimum width is called the “diffraction limit.”

This diffraction limit poses a limit on the resolution that can be obtained in semiconductor chip design and manufacture using the conventional Classical optical lithography technology. Because of this limit on the resolution, there is a physical limit on the number of transistors or other components that can be packed into a unit area of a chip, thereby placing a physical limit on the miniaturization that can be obtained.

FIG. 4 shows a schematic picture of a microscope used for conventional lithography. A classical light source is used to make a reduce-sized image of a complicated pattern, for example a lithography pattern for building up p-n junctions of millions of transistors, on the surface of a silicon chip. The resolution of the reduced image cannot be better than half of the wavelength of the classical light source λ/2, due to the diffraction effect. In other words, to this limit, one cannot reduce the size of the image any more. How to improve the spatial resolution? Classically, the only choice is to reduce the wavelength of the light. However, when the wavelength is too short, for example to the X-ray region, the optical microscope will stop working. There are no effective lenses working at such short wavelengths.

The disclosed teachings are aimed at overcoming the above noted problems in conventional lithography.

### SUMMARY

To realize the advantages and to overcome the disadvantages noted above, there is provided a microscopic image product comprising a light source that produces light that is made of entangled photons.

In another specific enhancement, the image product comprises a lithography microscope.

In another specific enhancement, the image product further comprises an optical imaging device for making reduced-size image.

In a more specific enhancement, the optical imaging device further comprises a first set of lenses that makes a Fourier transform of an image; and

a second set of lenses that retransforms the Fourier transform to a reduced-size image.

In an even more specific enhancement the image is a part of a semi-conductor chip manufacture.

In yet another specific enhancement the entangled photons are produced by nonlinear optical interactions and other optical processes.

In a more specific enhancement an entanglement condition for quantum lithography is required such that a diverging angle between entangled photons is substantially smaller than an angle which is equal to a distance between neighboring lines of the object pattern divided by a distance between the light source and the pattern.

Another aspect of the present invention is a chip manufacturing system comprising a substrate on which a thin photosensitive film which is sensitive only to multi-photon transition is deposited; a semiconductor chip; a light source generating entangled photon light; and a semiconductor design image pattern; wherein the chip manufacturing system is adapted to produce a substantially reduced size image pattern using the entangled photon light and wherein the substantially reduced image pattern is used in generating the semiconductor chip using the substrate with the thin film.

Yet another aspect of the present invention is a method of manufacturing a chip comprising generating entangled multi-photons. The entangled multi-photons are used to generate a microscopic image of an image of a semiconductor chip design. The microscopic image is impinged onto a semiconductor substrate with a photosensitive thin film that is sensitive only to multi-photon transition deposited on it. Further processing is performed to create the chip.

### BRIEF DESCRIPTION OF THE DRAWINGS

The above advantages of the disclosed teachings will become more apparent by describing in detail preferred embodiments thereof with reference to the attached drawings in which:

FIGS. 1(a)-(b) show schematically an example implementation illustrating the physics behind entangled quantum diffraction.

FIG. 2 shows an example implementation illustrating a folded version of FIG. 1.

FIG. 3 shows results illustrating a comparison of classical and quantum entangled photon lithography.

FIG. 4 shows a schematic picture of a microscope used for conventional lithography.

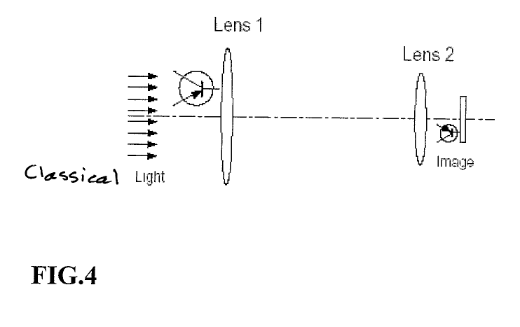

FIG. 5 shows a simple example setup for a semiconductor manufacturing system using quantum entangled light.

### IV. DETAILED DESCRIPTION

Synopisis

This disclosure teaches quantum lithography. Utilizing the entangled nature of a two-photon state, the limits paced by the classical diffraction limit is beaten at least by a factor of 2. Further, this is a quantum mechanical two-photon phenomenon that does not violate the uncertainty principle.

As noted above, classical optical lithography technology faces a limit due to the diffraction effect of light. This classical limit can be surpassed, surprisingly, by utilizing the quantum nature of entangled multi-photon states <1>. The minimum width of the entangled N-photon diffraction pattern is N times narrower than the width of the corresponding classical diffraction pattern. It should be noted that the present disclosure discusses the 2-photon entangled state in greater detail. However, this is only by way of example and should not be construed to be limiting. The scope of the disclosed teaching includes any N-photon entangled photon systems where N is any positive integer equal to or over 2.

Boto et al. <2>, and by Scully from a different approach <3>discuss the general theory of photon entanglement.

By way of example, and not by way of limitation, consider two-photon entangled states. For a two-particle maximally entangled EPR state, the value of an observable is undetermined for either single subsystem. However, if one subsystem is measured to be at a certain value for an observable, the value of that observable for the other subsystem is determined with certainty <1>. Because of this peculiar quantum nature, the two-photon diffraction pattern can be narrower, under certain conditions, than the one given by the classical limit. This effect has been experimentally observed by Kim and Shih <4>.

Quantum lithography is a topic that has recently attracted much attention. Classical optical lithography technology is facing its limit due to the diffraction limit. However, the classical limit can be surpassed by utilizing the quantum nature of entangled N-photon states. The spatial resolution of the lithography imaging using the entangled N-photon state is N times higher than that of the classical limit.

Using quantum the optical wavelength is thus maintained, but an N-photon entangled state is used thereby resulting in spatial resolution equivalent to that produced using a classical light with wavelength λ/N.

Comparison of Resolution Using Classical and Multi-Photon State

To demonstrate the quantum lithography idea experimentally, one could compare the spatial resolution of a microscope image by using classical and entangled multi-photon state. To have a clear demonstration, the experiment has to be done in a clever way. The interference-diffraction pattern of single or double-slit was measured on the Fourier transform plane (or far-field) of a lens. As is well-known, the first lens of a lithography microscope makes a Fourier transform of the “object”, which in this case is an image of a semiconductor design, and the second lens transforms it back to a reduce-sized image. On measuring the Fourier transform of the “object” and observing that the Fourier transform for the N-photon entangled light of wavelength λ is equivalent to that of using a classical light of λ/N instead of λ, it can be seen immediately that the spatial resolution of the reduce-sized image obtained by the second lens will be N times better.

Using two-photon entangled light source with wavelength λ results in a spatial resolution equivalent to using a classical light of λ/2 was obtained thereby beaten the diffraction limit of classical lithography a factor of 2.

Example Implementation

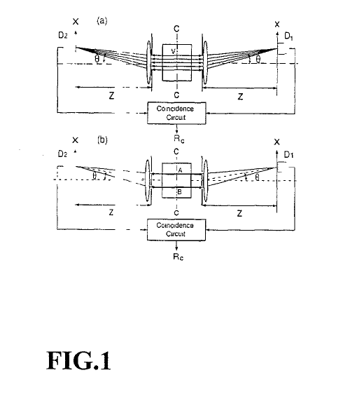

As noted above, the disclosed teaching uses the entangled nature of an N-particle system. The physics can be understood using the schematic example implementation illustrated in FIG. 1(a). An entangled photon pair is generated anywhere in region V; however, photons belonging to the same pair can only propagate (1) oppositely and (2) almost horizontally (quantitative discussion will be given later) as indicated in the figure. Two slits are placed symmetrically on the left and right sides of the entangled photon source. A photon counting detector is placed into the far-field zone (or the Fourier transform plane, if lenses are placed following the slits) on each side, and the coincidences between the “clicks” of both detectors are registered. The two detectors are scanning symmetrically, i.e., for each coincidence measurement, both detectors have equal x coordinates. A two-photon joint detection is the result of the superposition of the two-photon amplitudes, which are indicated in the figure by straight horizontal lines <6>. To calculate two-photon diffraction, all possible two-photon amplitudes are superposed.

FIG. 1 shown an example schematic of a two-photon diffraction-interference. The right and left sides of the picture represent the subsystems of an entangled pair. Detectors D1, D2 perform the join detection (coincident) measurement.

Unlike the classical case, a double integral is necessary involving the two slits and the two-photon amplitudes (parallel lines in FIG. 1). The two-photon counterpart of the classical intensity, the joint detection counting rate, is now sinc²(2β), which gives a distribution narrower than the classical pattern by a factor of 2.

To obtain a devise for performing quantum lithography, the symmetrical left and right sides of the setup descried above is folded together and the two independent detectors are replaced with a film that is sensitive only to two-photon light (two-photon transition material). This apparatus is an example apparatus implementation of a two-photon lithography system.

If one replaces the single slit in the setup shown in FIG. 1(a) with a double slit, FIG. 1(b), it can be seen that under the half-width diffraction pattern, the double-slit two-photon spatial interference pattern has a higher modulation frequency, as if the wavelength of the light were reduced to one-half. To observe the two-photon interference, one has to “erase” the first-order interference by reinforcing an experimental condition: δθ>λ/b, where δθ is the divergence of the light, b is the distance between the two slits, and λ is the wavelength.

A significant component of the above describe setup is a special two-photon source. The pair has to be generated in such a desired entangled way as described above. Under certain conditions, the two-photon state generated via spontaneous parametric down-conversion (SPDC) satisfies the above requirements. The working principle, as well as another example implementation is provided.

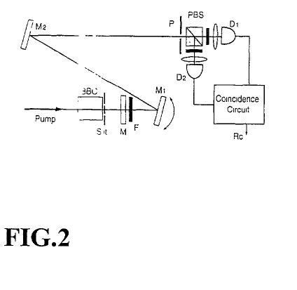

The schematic setup is illustrated in FIG. 2. It is basically the “folded” version of the double-slit interference-diffraction experiment shown in FIG. 1(b). The 458 nm line of an argon ion laser is used to pump a 5 mm BBO (β-0BaB₂O₄) crystal, which is cut for degenerate collinear type-II phase matching <7,8> to produce pairs of orthogonally polarized signal (e ray of the BBO) and idler (o ray of the BBO) photons. Each pair emerges from the crystal collinearly, with ωⱼ≅ωₚ/2, where ωⱼ(j=s, i ) are the frequencies of the signal and idler, respectively. The pump is then separated from the signal-idler pair by a mirror M, which is coated with reflectivity R≅1 for the pump and transmissivity T≅1 for the signal and idler.

For further pump suppression, a cutoff filter F is used. The signal-idler beam passes through a double slit, which is placed close to the output side of the crystal, and is reflected by two mirrors, M₁ and M₂, onto a pinhole P followed by a polarizing beam splitter PBS. The signal and idler photons are separated by PBS and are detected by the photon counting detectors D₁ and D₂, respectively. The output pulses of each detector are sent to a coincidence counting circuit with a 1.8 ns acceptance time window for the signal-idler joint detection. Both detectors are preceded by 10 nm bandwidth spectral filters centered at the degenerate wavelength, 916 nm. The whole block containing the pinhole, PBS, the detectors, and the coincidence circuit can be considered as a two-photon detector. Instead of moving two detectors together as indicated in FIG. 1, we rotate the mirror M₁ to “scan” the spatial interference-diffraction pattern relative to the detectors.

One important point to be emphasized is that the double slit must be placed as close as possible to the output surface of the BBO crystal. Only in this case, the observed diffraction pattern can be narrower than in the classical case by a factor of 2; see Eq. (9). Otherwise, it will be close to √2 as suggested in Ref. <3>.

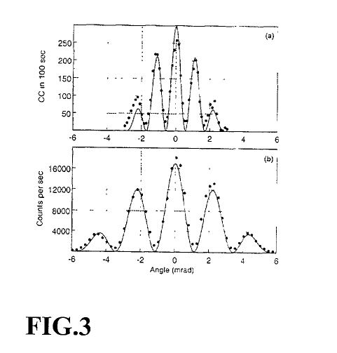

FIG. 3 reports the results using the above setup. In our experiment, the width of each slit is a=0.13 mm. The distance between the two slits is b=0.4 mm. The distance between the double slit and the pinhole P is 4 m. FIG. 3(a) shows the distribution of coincidences versus the rotation angle θ of mirror M₁. The spatial interference period and the first zero of the envelope are measured to be 0.001 and ±0.003 radians, respectively.

For comparison, the first-order interference-diffraction pattern of a classical light with 916 nm wavelength by the same double slit in a similar setup is shown in FIG. 3(b). The spatial interference period and the first zero of the envelope are measured to be 0.002 and ±0.006 radians, respectively.

FIG. 3.(a) shows results of measurement of the coincidences for the two-photon double-slit interference-diffraction pattern. FIG. 3(b) shows results of measurement of the interference-diffraction pattern for classical light in the same experimental setup. With respect to the classical case, the two-photon pattern has a faster spatial interference modulation and a narrower diffraction pattern width, by a factor of 2.

In both “classical” and “quantum” cases, similar standard Young's two-slit interference-diffraction patterns, sinc²[(πa/λ)θ] cos² [(πb/λ)θ] were obtained; however, whereas the wavelength for fitting the curve in FIG. 3(b) (classical light) is 916 μm, for the curve in FIG. 3(a) (entangled two-photon source) it has to be 458 μm. Clearly, the two-photon diffraction “beats” the classical limit by a factor of 2.

To further ensure that the effect of the SPDC photon pair with wavelength of 916 nm were observed but not the pump laser beam with wavelength of 458 nm, the BBO crystal is removed or rotated 90° to a non-phase-matching angle and the coincidence counting rate is examined. The coincidences remain zero during the 100 sec period, which is the data collection time duration for each of the data points, even in high power operation of the pump laser. Comparing this with the coincidence counting rate obtained with BBO under phase matching, see FIG. 3(a), there is no doubt that the observation is the effect due to the SPDC photon pairs.

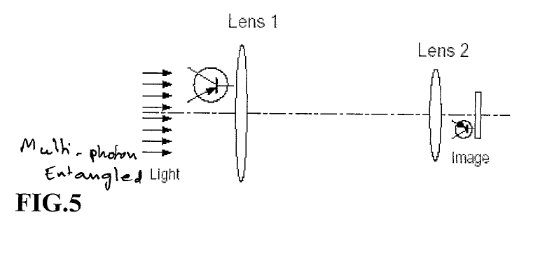

FIG. 5 shows a simple example setup for a semiconductor manufacturing system using quantum-entangled light.

Explanation of Results

To explain the result, the quantum nature of the two-photon state has to be taken into account. SPDC is a nonlinear optical process in which pairs of signal-idler photons are generated when a pump laser beam is incident onto an optical nonlinear material <7,8>. Quantum mechanically, the state can be calculated by the first-order perturbation theory <7>and has the form

$$⌊ Ψ ⟩ = {\sum }_{si} F ( {ω}_{s} , {ω}_{i} , {k}_{s} , {k}_{i} ) {a}_{s}^{t} [ ω ( {k}_{s} ) ] {a}_{i}^{t} [ ω ( {k}_{i} ) ] \vert 0 ⟩ , \qquad ( 1 )$$

where ωⱼ, kⱼ(j=s, i, p) are the frequencies and wave vectors of the signal (s), idler (i), and pump (p), respectively, F (ωₛ, ωᵢ, kₛ, kᵢ) is the so-called biphoton amplitude, and aₛ and aᵢ are creation operators for the signal and idler photons, respectively. The pump frequency ωₚ and wave vector kₚ can be considered as constants. The biphoton amplitude contains δ functions of the frequency and wave vector,

F(ωₛ, ωᵢ, kₛ, kᵢ) ∝δ (ωₛδ (ωₛ+ωᵢ−ωₚ) xδ (kₛ+kᵢ−kₚ) (2)

The signal or idler photon could be in any mode of the superposition (uncertain); however, due to Eq. (2), if one photon is known to be in a certain mode then the other one is determined with certainty.

The phase-matching conditions resulting from the δ functions in Eq. (2),

ωₛ+ωᵢ=ωₚ, kₛ+kᵢkₚ, (3)

play an important role in the experiment. The transverse component of the wave vector phase-matching condition requires that

kₛ sinαₛ=kᵢ sinαᵢ, (4)

where αₛ and αᵢ are the scattering angles inside the crystal. Upon exiting the crystal, Snell's law thus provides

ωₛ sinβₛ=ωᵢ sinβᵢ, (5)

where βₛ and βᵢ are the exit angles of the signal and idler with respect to the kₚ direction. Therefore, in the degenerate case, the signal and idler photons are emitted at equal, yet opposite, angles relative to the pump, and the measurement of the momentum (wave vector) of the signal photon determines the momentum (wave vector) of the idler photon with unit probability and vice versa. In the collinear case, as in the setup describe above, the scattering angles of the signal and idler photons are close to zero and occupy the range Du, which is determined by the size of both the crystal and the pump beam; see <9>.

The coincidence counting rate R_{c} is given by the probability P₁₂ of detecting the signal-idler pair by detectors D₁ and D₂ jointly,

$$\begin{matrix}{P}_{12} = ⟨ Ψ \vert {E}_{1}^{( - )} {E}_{2}^{( + )} {E1}^{( + )} \vert Ψ ⟩ \\ = {\vert ⟨ 0 \vert {E}_{2}^{( + )} {E}_{1}^{( + )} \vert Ψ ⟩ \vert}^{2} ,\end{matrix} \qquad ( 6 )$$

where |Ψ> is the two-photon state of SPDC and E₁, E₂ are fields on the detectors. The effect of two-photon Young's interference can be easily understood if the signal and idler photons are always assumed to go through the same slit and never go through different slits. This approximation holds if the variation of the scattering angle inside the crystal satisfies the condition:

Δθ<<b/D, (7)

where D is the distance between the input surface of the SPDC crystal and the double slit. In this case, the state after the double slit can be written

|ψ>=|0>αᵢ^{\}exp(iφ_{A})+bₛ^{\}bᵢ^{\}exp(iφ_{B})]|0>,

as

where ε<<1 is proportional to the pump field and the nonlinearity of the crystal, φ_{A} and φ_{B} are the phases of the pump field at region A (upper slit) and region B (lower slit), respectively, and aⱼ⁺, bⱼ⁺ are the photon creation operators for photons passing through the upper slit (A) and the lower slit (B), respectively. In the setup secribed above, the ratio (b/D)/Δθ≅b 30 and Eq. (7) are satisfied well enough. Moreover, even the ratio (a/D)/Δθ is of the order of 10, which satisfies the condition for observing two-photon diffraction:

Δθ<<a/D (9)

In Eq. (6), the fields on the detectors are given by E₁⁽⁺⁾=αₛexp(ikr_{A1})+bₛexp(ikr Bi 1I

E₂⁽⁺⁾=αᵢexp(ikr_{A2})+b₁exp(ik 2)+b, exp(iktB, I-[

where r_{Ai} (r_{Bi}) are the optical path lengths from region A (B) to the ith detector. Substituting Eqs. (8) and (10) into Eq. (6), we get

$${R}_{c} ∝ {P}_{12} = {ε}^{2} {\vert exp ( {ikr}_{A} + i {ϕ}_{A} ) + exp ( {ikr}_{B} - i {ϕ}_{B} ) \vert}^{2} ∝ 1 + cos [ k ( {r}_{A} - {r}_{B} ) ] , \qquad ( 11 )$$

where r_{A}≡r_{A1}+r_{A2} (r_{B}≡r_{B1}+r_{B2}) and φ_{A}=φ_{B} in Eq. (11).

In the far-field zone (or the Fourier transform plane), interference of the two amplitudes from Eq. (8) gives

R_{c}(θ) ∝cos²[2πb/λ)θ)] (12)

Equation (12) has the form of a standard Young's two-slit interference pattern, except having the modulation period one-half of the classical case or an equivalent wavelength of λ/2.

To calculate the diffraction effect of a single slit, an integral of the effective two-photon wave function over the slit width is needed. Quite similarly to Eq. (12), it gives

R_{c} (θ)∝sinc²[2πa/λ)θ)] (13)

Equation (13) has the form of a standard single-slit diffraction pattern, except having one-half of the classical pattern width.

The combined interference-diffraction coincidence counting rate for the double-slit case is given by

Rc (θ)∝sinc²[2πa/λ)θ)cos²[(2πb/λ)θ], (14)

which is a product of Eqs. (12) and (13).

The experimental observations have confirmed the above quantum mechanical predictions.

In conclusion, significant advantages can be seen, specifically in the case of a large number of entangled particle states. Based on an entangled N-photon scheme one can beat the classical limit by a factor of N, which is equivalent of using shorter wavelength of λ/N, however, keep the wavelength of λ. This is a quantum mechanical N-photon phenomenon but not a violation of the uncertainty principle.

Other modifications and variations to the invention will be apparent to those skilled in the art from the foregoing disclosure and teachings. Thus, while only certain embodiments of the invention have been specifically described herein, it will be apparent that numerous modifications may be made thereto without departing from the spirit and scope of the invention.

What is claimed is:

1. A microscopic image product comprising:
a light source that produces light that is made of entangled photons.

2. The microscopic image product of claim 1, wherein the image product comprises a lithography microscope.

3. The microscopic image product of claim 1, wherein the image product further comprises an optical imaging device for making reduced-size image.

4. The microscopic image product of claim 3 wherein the optical imaging device further comprises:
a first set of lenses that makes a Fourier transform of a semiconductor design pattern; and
a second set of lens that retransforms the Fourier transform to a reduced-size pattern.

5. The microscopic image product of claim 3 wherein the image is a part of a semi-conductor chip manufacture.

6. The microscopic image product of claim 1 wherein the entangled photons are produced by nonlinear optical interactions and other optical processes.

7. The microscopic image product of claim 6 wherein an entanglement condition for quantum lithography is required such that a diverging angle between entangled photons is substantially smaller than an angle which is equal to a distance between neighboring lines of the object pattern divided by a distance between the light source and the pattern.

8. A chip manufacturing system comprising:
a substrate on which a thin photosensitive film that is sensitive only to a multi-photon transition is deposited;
a semiconductor chip;
a light source generating multi-photon entangled photon light; and
a semiconductor design pattern;
wherein the chip manufacturing system is adapted to produce a substantially reduced size image of the semiconductor design pattern using the entangled photon light and
wherein the substantially reduced image pattern is used in generating the semiconductor chip using the substrate with the thin film.

9. The chip manufacturing system of claim 8 wherein the system further comprises an optical imaging device for making reduced-size image.

10. The chip manufacturing system of claim 9, wherein the optical imaging device further comprises:
a first set of lenses that makes a Fourier transform of the semiconductor design pattern image; and
a second set of lenses that retransforms the Fourier transform to a reduced-size image.

11. The chip manufacturing system of claim 8 wherein the entangled photons are produced by nonlinear optical interactions and other optical processes.

12. The chip manufacturing system of claim 11 wherein an entanglement condition for quantum lithography is required that the diverging angle between entangled photons is substantially smaller than an angle which is equal to the distance between neighboring lines of the object pattern divided by the distance between the light source and the pattern.

13. A method of manufacturing a chip comprising;
generating entangled multi-level photons;
using the entangled multi-level photons to generate a microscopic image of an image of a semiconductor chip design pattern;
impinging the microscopic image onto a semiconductor substrate with a photosensitive thin film that is sensitive only to multi-photon transition deposited on it; and
performing further processing to create the chip.

14. The method of claim 13 wherein the entangled photons are produced by nonlinear optical interactions and other optical processes.

15. The method of claim 13 wherein an entanglement condition for quantum lithography is required such that a diverging angle between entangled photons is substantially smaller than an angle which is equal to the distance between neighboring lines of the object pattern divided by the distance between the light source and the pattern.

16. The method of claim 13 wherein the step of impinging includes:
defining dividing boundaries in the thin film layer to form a plurality of tiles between the dividing boundaries in a precise pattern.

17. The method of claim 13 further comprising the step of:
removing a subset of the tiles to form the microscopic image in the thin film.
